(19) United States
(12) Patent Application Publication
(10) Pub. No.: US 2023/0106323 A1
(43) Pub. Date: Apr. 6, 2023

(54) Computer Assisted Surgery Device Having A Robot Arm And Method For Operating The Same

(71) Applicant: Stryker European Operations Limited, Carrigtwohill (IE)

(72) Inventors: Jakob Alexander Kemper, Heemstede (NL); Lars Metz, Kiel (DE)

(21) Appl. No.: 17/909,938

(22) PCT Filed: Mar. 19, 2020
(86) PCT No.: PCT/IB2020/052518

§ 371 Date: Sep. 7, 2022

(51) Int. Cl. A61B 34/10; A61B 34/20; A61B 34/30

(52) U.S. Cl. CPC A61B 34/10; A61B 34/20; A61B 34/30; A61B 2034/102; A61B 2034/305; A61B 2034/107; A61B 2090/3966

(57) ABSTRACT

A computer assisted surgery device and a method for operating the same which allows a more efficient positioning and application of an implant with respect to a bony structure, wherein the computer-assisted surgery device having a robot arm and a method for operating the same resulting in a shorter operation time and less intensity of x-ray exposure for a patient.

### FIELD OF THE INVENTION

The present invention relates to a computer assisted surgery device and a method for operating the same which allows a more efficient positioning and application of an implant with respect to a bony structure, and in particular to a computer-assisted surgery device having a robot arm and a method for operating the same resulting in a shorter operation time and less intensity of x-ray exposure for a patient.

### BACKGROUND OF THE INVENTION

For applying an implant to a body of a patient, in particular to a bony structure of a patient, it is required to make sure, that the implant position with respect to the bony structure is correct, so that the patient does not suffer from a misalignment of the implant. This is in particular relevant for implants for legs, as a misalignment of implants in the leg, in particular on the hip, the knee or the ankle, may result in a serious limitation of patient's mobility, in particular the walking capabilities.

Applying an implant to a patient, in particular to a bony structure of a patient, therefore requires a sufficient monitoring of the application and the alignment of the implant with respect to the bony structure. In the past, this was achieved by a repeated monitoring of the situation by taking a plurality of x-ray images illustrating the present implanting situation between the implant and the bony structure, so that the surgeon based on the respective x-ray imaging could re-align the implant to the bony structure repeatedly unless the implant has arrived at the desired position and alignment with the bony structure. This may lead to a repeated, i.e. higher x-ray exposure of the patient, which may have undesired effects for the patient.

In the past, it was also usual for a surgeon to place a K-wire tip to the outside of the bone or to insert the K-wire a few cm, so that a better illustration was possible. Nevertheless, also this approach required a large number of iterations including a large number of x-ray images with a high radiation and at the same time a limited accuracy.

In the past, also applications were known where a full navigation system was used which requires trackers on the relevant bone fragments and instruments as well as a full navigation setup with a stereo camera, which also resulted in a high x-ray exposure.

In view of the above, there was a need for providing a device and a method for operating the device allowing a high accuracy and a reduced x-ray exposure over the previously known devices and methods.

### SUBJECT-MATTER OF THE PRESENT INVENTION

The present invention provides a computer assisted surgery device and a method for operating the same according to the independent claims. Further embodiments are incorporated in the dependent claims.

According to an aspect of the invention, there is provided a method for operating a computer assisted surgery device comprising acquiring an x-ray image of a bony structure together with at least one of an implant targeting device having an implanting trajectory and a reference geometry, and an implant having an implanting trajectory and a reference geometry, the bony structure having an implanting area with a predetermined implanting axis; acquiring a deviation of the implanting trajectory of at least one of the implant targeting device and the implant from the predetermined implanting axis of the implanting area based on the acquired x-ray image; determining a measure of a required motion of the at least one of the implant targeting device and the implant to be executed for bringing the implanting trajectory of the at least one of the implant targeting device and the implant from a deviated state of the implanting trajectory of the at least one of the implant targeting device and the implant into alignment with the predetermined implanting axis of the implanting area; and controlling a motion of at least one of the implant targeting device and the implant based on the determined measure of required motion.

This allows to determine the relative position of an implant or/and its reference geometry with respect to a bony structure and to determine the deviation between a present implanting trajectory and a desired implanting trajectory which should correspond to an implanting axis of an implanting area of a bony structure, based on the acquired x-ray image. The method determines the required motion of the implant to move the same from the present trajectory to the desired trajectory and to control this motion based on the determined measure of required motion. In particular, the method does not rely on a trial and error approach of the surgeon, but on the analysis of an x-ray image, so that the determination of the deviation of the present and the desired implanting trajectory, as well as the determination of the required motion is no longer done by the surgeon, but supported by the method when operating the computer assisted surgery device. This reduces the human impact on a re-alignment as well as the number of repeated x-ray images for monitoring the respective alignment steps. It can be expected, that the method for operating the computer assisted surgery device is more exact than the manual work of the surgeon and may also be faster than the surgeon which will reduce the duration of the incision and increase the accuracy. Further, it can be expected, that repeated x-ray imaging is no longer required, so that the x-ray exposure to the patient will be lower when applying the method for operating the computer assisted surgery device. Controlling the motion may include providing a surgeon with information how the implant targeting device or the implant is to be moved, but may also include controlling actuators to do so.

According to an embodiment of the invention, the computer assisted surgery device has a segmented robot arm with a plurality of segments, wherein two adjacent segments are coupled with a joint being capable of being controllably brought from a fixed state into a released state and vice versa, wherein the segmented arm with one end is connected to a fix point and with the other end is connected to the implant targeting device and/or the implant, wherein the method further comprises before acquiring the x-ray image, bringing the joints of the segmented arm into the fixed state; after determining a measure of required motion, bringing at least a part of the joints into the released state; controlling a motion of at least a part of the segments adjacent to the released joints in order to move the at least one of the implant targeting device and the implant according to the determined measure of required motion; and then optionally bringing the joints from the released state into the fixed state.

Thus, it is possible to control a robot arm, in particular by controlling the movability of the robot arm during the method for operating the computer assisted surgery device. The fix point may be a point which is used as a reference point related to which the implant and/or its reference geometry can be moved. Also the position of the patient, in particular the patient's bony structure to which the implant is to be applied may be defined with respect to the fix point, so that the relative position of the implant and the bony structure can be determined. The surgeon may bring the segmented arms of the device for computer assisted surgery, e.g. segments of the robot arm into an initial position which is e.g. close to an expected implanting situation. The method then brings the joints of the segmented arm of the robot arm into a fixed state in order to create a defined geometrical situation for which an x-ray image can be taken as a basis for the acquisition of the deviation and the controlling of the segmented robot arm. When the x-ray image was taken and a deviation was acquired and the measure of required motion was determined, the method controls the segmented robot arm in order to bring at least a part of the joints into the released state, so that the method may control a motion of the at least a part of segments adjacent to the released joints to move them into the desired and required position corresponding to the required motion. As an alternative, the method may provide the surgeon with information how to move the segmented arm when the joints are released. For this purpose a system which carries out the method may provide the surgeon with various coordinates for how far to move the targeting device and/or the implant. The entire situation may be monitored continuously, periodically or by incident. The monitoring of the situation may be updated on a monitor to show the real time position. The surgeon may be provided with a feedback how exact the surgeon meets the calculated position, e.g. by a stepped warning, e.g. by a green light, orange light, and red light, depending on how exact the surgeon works. The surgeon may also receive a haptic feedback, which e.g. generates a recognizable resistance when leaving the calculated position to a certain extend. Then, the method controls the segmented robot arm to bring the joints from the released state into the fixed state in which the deviation of the implanting trajectory of the implant targeting device and the implant is below a threshold and which corresponds to a sufficient implanting situation. The method may determine whether the deviation is below a threshold. This may be done by comparing the calculated coordinates with the actual coordinates. The threshold may be pre-determined based on an implant database or bone database, and may be pre-set to a certain percentage of e.g. bone geometries in the bone database, so that the threshold covers e.g. 95% of all relevant bone geometries. In case the deviation is above the threshold, the method may re-iterate the process. This may also apply, if the surgeon decides to not accept the position which was generated by the method, i.e. in case the actual operation situation changes upon a recent diagnostic of the surgeon during the incision.

It should be noted, that after bringing the joints from the released state into the fixed state, optionally an additional x-ray image may be taken in order to monitor, whether the determined measure of a required motion was sufficient for compensating the deviation of the implanting trajectory of the implant targeting device and the implant from the predetermined implanting axis of the implanting area. If this is the case, no further steps may be required. However, if the acquired deviation after the second x-ray image is larger than the desired threshold, the previous steps may be repeated unless the deviation is below a desired threshold.

According to an embodiment of the invention, the segmented robot arm has a plurality of actuators each being adapted to controllably actuate a motion of two adjacent segments with respect to each other along their connecting joint, wherein the method further comprises before acquiring the x-ray image controlling the actuators to bring the joints of the segmented arm into the fixed state; after determining a measure of required motion, controlling the actuators to move at least a part of the segments to move the at least one of the implant targeting device and the implant according to the determined measure of required motion; and controlling the actuators to bring the joints of the segmented arm into the fixed state.

Thus, the segmented robot arm may be provided as an integral entity having actuators for actuating the segments and no external entity is required for actuating the segments of the robot arm. Thus, the surgeon does not need to actuate the segments any longer.

According to an embodiment of the invention, acquiring the x-ray image of the bony structure together with at least one of the implant targeting device and the implant comprises acquiring a first at least bi-planar image from a first point of view onto the bony structure together with the at least one of the implant targeting device and the implant and a second at least bi-planar image from a second point of view onto the bony structure together with the at least one of the implant targeting device and the implant; correlating the first at least bi-planar image and the second at least bi-planar image; and generating a three dimensional image of the bony structure, optionally together with the implant, based on the first at least bi-planar image, the second at least bi-planar image, and the correlation of the first at least bi-planar image and the second at least bi-planar image.

Thus, by acquiring and generating a three dimensional image, a more exact determination of a required motion is possible, so that the method for operating a computer assisted surgery device results in a more exact positioning of an implant and/or an implant targeting device with respect to a bony structure. The further dimension by taking the first and second x-ray image from different points of view may allow to identify artefacts in a single x-ray image and its misinterpretation.

According to an embodiment of the invention, the method further comprises determining the implanting area and the predetermined implanting axis based on the acquired x-ray image of the bony structure and a bone data base having stored therein a plurality of bony structures, optimized implanting areas thereon and/or therein, and predetermined implanting axes, as well as a correlation thereof.

Thus, the method may operate the computer assisted surgery device much quicker, as some of the structures may be taken from a knowledge data base, which limits the variety. Further, a certain likelihood may be applied to arrive at usual bone geometries, as well as implant geometries as well as correlations thereof.

According to an embodiment of the invention, an implanting area of a bony structure represents an area which is defined by a geometry of an implant to be implanted on and/or in the bony structure.

Thus, the bony structure may be defined by the geometry of the implant and the desired orientation of the implant with respect to the bony structure. This may also consider a bending of an implant. In particular cases the implant may bend when implanting the implant, so that the expected bending may be considered when defining the implanting area.

According to an embodiment of the invention, the method comprises after controlling a motion of the at least one of the implant targeting device and the implant, acquiring a further at least bi-planar image from a first view point onto the bony structure together with the at least one of the implant targeting device and the implant, and a further at least bi-planar image from a second point of view onto the bony structure together with the at least one of the implant targeting device and the implant; determining a deviation of the implanting trajectory of the at least one of the implant targeting device and the implant from the predetermined implanting axis of the implanting area of the bony structure; and if the deviation is above a predetermined threshold, repeating determining a measure of a required motion of the at least one of the implant targeting device and the implant to be executed for bringing the implanting trajectory from an acquired deviation of the implanting trajectory of the at least one of the implant targeting device and the implant into alignment with the implanting axis of the implanting area of the bony structure and controlling a motion of the at least one of the implant targeting device and the implant.

Thus, a repeated iterative alignment of the implant and/or implant targeting device with respect to the bony structure can be achieved, which may further improve the accuracy of the alignment of the implant and/or implant targeting device with respect to the bony structure. Even if the deviation is below a predetermined threshold, this may be used to confirm the correct determination of the required motion and the correct execution of the motion of the implant targeting device and/or the implant with respect to the bony structure. Further, the repeated imaging may be applied in case the surgeon modifies the implanting axis upon inter operative diagnosis.

According to an embodiment of the invention, the method is carried out by a computer having a memory and a processor, wherein the computer is adapted for carrying out the method, in particular the method steps, in particular acquiring a deviation of the implanting trajectory from the predetermined implanting axis based on an acquired x-ray image, determining a measure of a required motion for bringing the implanting trajectory into alignment with the predetermined implanting axis, and controlling a motion of at least one of the implant targeting device and the implant.

Thus, the method can be supported by a computer. The computer does not need to be implemented in a single device, but may also be a distributed computer, where processors and/or memories may be located at different places, e.g. in the operating theatre and on a server. In particular a database for bone and implant geometries as well as computational intensive operations may be realized on a remote server computer, whereas the other operations like manipulating the robot arm to move into its proper location may be realized on an on-site computer unit.

According to a further aspect of the invention, there is provided a device for computer assisted surgery, the device comprises an image acquiring unit being adapted for acquiring an x-ray image of a bony structure together with an implant targeting device having an implanting trajectory and a reference geometry; a deviation acquiring unit being adapted for acquiring a deviation of the implanting trajectory of the implant targeting device from a predetermined implanting axis of an implanting area of the bony structure based on the acquired x-ray image; a motion determining unit being adapted for determining a measure of a required motion of the implant targeting device to be executed for bringing the implanting trajectory of the implant targeting device from the acquired deviation of the implanting trajectory of the implant targeting device into alignment with the predetermined implanting axis of the implanting area; and a motion controlling unit being adapted for controlling a motion of the implant targeting device based on the measure of required motion determined by the motion determining unit.

Thus, a device may be provided, which allows a more exact and faster application of an implant with respect to a bony structure and to reduce an x-ray exposure to a patient according to the method as described above.

According to an aspect of the invention, the device comprises an image acquiring unit being adapted for acquiring an x-ray image of a bony structure together with an implant having an implanting trajectory and a reference geometry; a deviation acquiring unit being adapted for acquiring a deviation of the implanting trajectory of the implant from a predetermined implanting axis of an implanting area of the bony structure based on the acquired x-ray image; a motion determining unit being adapted for determining a measure of a required motion of the implant to be executed for bringing the implanting trajectory of the implant from the acquired deviation of the implanting trajectory of the implant into alignment with the predetermined implanting axis of the implanting area; and a motion controlling unit being adapted for controlling a motion of the implant based on the measure of required motion determined by the motion determining unit.

Thus, it is not only possible to bring an implant targeting device into the correct position for applying an implant to a bony structure, but to also bring an implant itself to the correct position of a bony structure according to the method as described above.

According to an aspect of the invention, there is provided a device for computer assisted surgery comprising a first segmented arm with a plurality of segments, wherein at least two adjacent segments are coupled with a joint being capable of being controllably brought from a fixed state into a released state and vice versa, wherein the segmented arm with one end is connected to a fix point and with the other end is connectable to at least one of an implant targeting device having an implanting trajectory and a reference geometry, and an implant having an implanting trajectory and a reference geometry; an image acquiring unit being adapted for acquiring an x-ray image of a bony structure together with the at least one of the implant targeting device and the implant at a fixed state of the first segmented arm; a deviation acquiring unit being adapted for acquiring a deviation of the implanting trajectory of the at least one of the implant targeting device and the implant from a predetermined implanting axis of an implanting area of the bony structure based on the acquired x-ray image; a motion determining unit being adapted for determining a measure of a required motion of the at least one of the implant targeting device and the implant to be executed for bringing the implanting trajectory of the at least one of the implant targeting device and the implant from the acquired deviation of the implanting trajectory of the at least one of the implant targeting device and the implant into alignment with the predetermined implanting axis of the implanting area; and a motion controlling unit being adapted for bringing the first segmented arm from the fixed state into the released state, for controlling a motion of the first segmented arm based on the measure of required motion determined by the motion determining unit, and for bringing the first segmented arm from the released state into the fixed state.

Thus, it is possible to provide a device, which has a segmented arm which can be brought, in a released state of its joints, into a desired position in order to hold an implant or an implant targeting device with respect to a bony structure. Fixing the joints allows to bring the segmented arm into a fixed state in order to acquire a reliable x-ray image. As the segmented arm can be brought again into a released state of its joints, it is possible to bring the segmented arm into a desired position for positioning the implant targeting device and/or the implant with respect to the bony structure based on an acquired deviation and a determined measure of a required motion, so that the motion of the segmented arm can be controlled to bring the implant and/or the implant targeting device into a position which is suitable for implanting the implant with respect to the bony structure.

According to an aspect of the invention, there is provided a device for computer assisted surgery comprising a first segmented robot arm having a plurality of segments and a plurality of actuators each being adapted to controllably actuate a motion of two adjacent segments with respect to each other along a connecting joint of two adjacent segments, wherein the joint being capable of being controllably brought from a fixed state into a released state and vice versa, wherein the first segmented robot arm with one end is connected to a fix point and with the other end is connectable to at least one of a implant targeting device having an implanting trajectory and a reference geometry, and an implant having an implanting trajectory and a reference geometry; an image acquiring unit being adapted for acquiring an x-ray image of a bony structure together with the at least one of the implant targeting device and the implant at a fixed state of the first segmented robot arm; a deviation acquiring unit being adapted for acquiring a deviation of the implanting trajectory of the at least one of the implant targeting device and the implant from a predetermined implanting axis of an implanting area of the bony structure based on the acquired x-ray image; a motion determining unit being adapted for determining a measure of a required motion of the at least one of the implant targeting device and the implant to be executed for bringing the implanting trajectory of the at least one of the implant targeting device and the implant from the acquired deviation of the implanting trajectory of the at least one of the implant targeting device and the implant into alignment with the predetermined implanting axis of the implanting area; and a motion controlling unit being adapted for bringing the connecting joints of the first segmented robot arm from the fixed state into the released state for controlling the motion of the actuators of the first segmented robot arm based on the measure of required motion determined by the motion determining unit, and for bringing the joints of the first segmented robot arm from the released state into the fixed state.

Thus, a device can be provided which is fully operable from a fixed state into a variable state to control the position and motion of segmented arms in the variable state and afterwards to again fix the segments with respect to each other in order to arrive at a fixed state. Actuators may be used to control the movement of the segmented arms when the joints are released, so that the device for computer assisted surgery may have an autarkic robot arm which can be controlled with respect to a fixed and released state as well as the motion of the segmented arms. As an alternative, the device may provide the surgeon with information how to move the segmented arm when the joints are released. For this purpose the device may provide the surgeon with various coordinates for how far to move the targeting device and/or the implant. The device may monitor the situation continuously, periodically or by incident. The monitoring of the situation may be updated on a monitor to show the real time position. The device may provide the surgeon with a feedback how exact the surgeon meets the calculated position, e.g. by a stepped warning, e.g. by a green light, orange light, and red light, depending on how exact the surgeon works. The device may also give to the surgeon a haptic feedback, e.g. by generating a recognizable resistance when leaving the calculated position to a certain extend. Then, the device may control the segmented robot arm to bring the joints from the released state into the fixed state in which the deviation of the implanting trajectory of the implant targeting device and the implant is below a threshold and which corresponds to a sufficient implanting situation. The device may determine whether the deviation is below a threshold. The device may compare the calculated coordinates with the actual coordinates. The device may use a pre-determined threshold which may be based on an implant database or bone database, and may be pre-set to a certain percentage of e.g. bone geometries in the bone database, so that the threshold covers e.g. 95% of all relevant bone geometries. In case the deviation is above the threshold, the device may re-iterate the process. The blocked or fixed state of the joints can be achieved by providing a locking structure, e.g. a clutch, in particular a blocking clutch or frictional clutch. The blocked or fixed state can also be achieved by using actors which when not moving are in a fixed state. The fixed state can also be achieved by automatically activating the clutch for fixing the joints as soon as the actuators have stopped moving the segments adjacent to the joint. It should be noted that beside the above described (first) segmented arm also a further second segmented arm may be provided for other assisted purposes.

According to an embodiment of the invention, the image acquiring unit is adapted for acquiring a first at least bi-planar image from a first point of view onto the bony structure together with the at least one of the implant targeting device and the implant, and a second at least bi-planar image from a second point of view onto the bony structure together with the at least one of the implant targeting device and the implant, and for composing an x-ray image out of the first at least bi-planar image and the second at least bi-planar image.

Thus, it is possible to create a three dimensional image of the implanting situation, which may simplify the determination of the deviation as well as the determination of the required motion to arrive at an implanting situation, wherein the implant and/or the implant targeting device is correctly positioned with respect to the bony structure. Further, with an x-ray image from a second point of view which differs from the first point of view, artefacts in an x-ray image may be identified and eliminated, and a misalignment based on such artefacts may be avoided.

According to an embodiment of the invention, the image acquiring unit comprises a correlation unit being adapted for correlating the first at least bi-planar image and the second at least bi-planar image and generating a three dimensional bony structure based on the first at least bi-planar image, the second at least bi-planar image, and the correlation of the first at least bi-planar image and the second at least bi-planar image.

Thus, it is possible to generate not only a three dimensional image, but also to generate three dimensional structures, in particular a three dimensional bony structure which allows a better determination of the deviation as well as the required motion to bring the implant and/or implant targeting device into a correct position for positioning the implant with respect to the bony structure.

According to an embodiment of the invention, the device further comprises an implanting area determining unit being adapted for determining the implanting area and the predetermined implanting trajectory based on the acquired bony structure and a bone data base having stored therein a plurality of bony structures, optimized implanting areas thereon and/or therein, and predetermined implanting axes, as well as a correlation thereof.

Thus, it is possible to reduce the calculation and computational effort, as the device may be provided with predetermined structures, so that the determination of the deviation as well as the determination of the required motion can be performed faster and more accurate.

According to an embodiment of the invention, the device further comprises as part of a system a reference geometry, wherein the reference geometry is a reference body which is attachable to at least one of the implant targeting device and the implant, thus representing the unique position and orientation of the implant targeting device and/or implant.

Thus, it is possible to derive the position and orientation of an implant targeting device by taking only one single x-ray shot, as the reference geometry has a particular shape, which illustrates the orientation and position of the reference geometry uniquely. Nevertheless, a second x-ray image from a different point of view may provide additional information and a higher redundancy so as to eliminate possible failures in image recognition form the x-ray images.

According to an embodiment of the invention, the device further comprises as part of a system an implant targeting device, wherein the reference geometry is an integral portion of the implant targeting device, wherein the integral portion has a geometry having a unique projection pattern for each projection direction.

Thus, it is possible to provide an implant targeting device, which because of the unique reference geometry, itself has an unique projection pattern for each projection direction, so that the orientation and position of the implant targeting device and/or implant connected thereto may be achieved by taking a single x-ray image.

According to an embodiment of the invention, the device further comprises as part of a system an implant, wherein the reference geometry is an integral portion of the implant, wherein the integral portion has a geometry having a unique projection pattern for each projection direction.

Thus, it is possible to determine the orientation and position of the implant itself by way of the reference geometry.

According to an embodiment of the invention, the reference geometry has a plurality of fiducial markers, wherein the fiducial markers have a spatial arrangement having a unique projection pattern for each projection direction.

Therefore, it is possible to determine the position and orientation of the reference geometry based on the plurality of fiducial markers which are distributed over the reference geometry in a unique and well-known pattern, so that from that pattern of fiducial markers it can be concluded based on the single x-ray image how the position and the orientation of the reference geometry is. In the same way, the position and orientation of an implant or an implant targeting device can be determined to which the reference geometry is coupled or formed as an integral portion.

### BRIEF DESCRIPTION OF THE FIGURES

FIG. 1 illustrates a schematic overview of a device for computer assisted surgery according to an exemplary embodiment of the invention.

FIG. 2 illustrates an implanting trajectory of an implant and an implanting axis of an implanting area and their relative position to each other to be handled with a device for computer assisted surgery according to exemplary embodiments of the invention.

FIG. 3 illustrates a schematic overview for taking a first bi-planar image and a second bi-planar image and its correlation to a three dimensional image according to an exemplary embodiment of the invention.

FIG. 4 illustrates a sectional view of an implanting trajectory and an implanting axis and their deviation.

FIG. 5 illustrates an implant targeting device having attached thereto a reference geometry with fiducial markers according to an exemplary embodiment of the invention.

FIG. 6 illustrates an implant having a reference geometry in form of a unique projection geometry according to an exemplary embodiment of the invention.

FIG. 7 illustrates an implant having a reference geometry in form of an included plurality of fiducial markers according to an exemplary embodiment of the invention.

FIG. 8 illustrates a device for computer assisted surgery while positioning an implant according to an exemplary embodiment of the invention.

FIG. 9 illustrates a schematic sequence of procedural steps of a method for operating a computer assisted surgery device according to an exemplary embodiment of the invention.

FIG. 10 illustrates a more detailed sequence of steps of a method for operating a computer assisted surgery device according to an exemplary embodiment of the invention.

Exemplary embodiments will be described in more detail with respect to the enclosed figures, where same or corresponding references refer to the same or corresponding elements and structures.

### DETAILED DESCRIPTION OF EXEMPLARY EMBODIMENTS

The present invention provides a computer assisted surgery system and method for operating the same that allows an easier positioning of implants with respect to bony structures. This device and method for operating the same also allow a support in positioning implants and sub-implants, as well as implants and tooling devices with respect to each other. The invention overcomes problems with pin and screw targeting, that in multiple indications require significant radiation, and in some cases requires multiple passes with a K-wire and further has a limited accuracy in case 3D information on the pin or screw position is normally not available in the operational room. As in the past, often iterative and free-hand targeting with fluoroscopy was applied, for some applications combined with temporary K-wires to verify position or a full navigation with bone trackers and a stereo camera, the drawbacks of such methods and devices can be overcome with the present invention.

The present invention provides a device and a method for operating the device, which constitutes a combination of a stereotactic method with a robotic arm arrangement for multiple targeting exercises in traumatology. The concept is to identify the relative spatial position of bony and anatomical structures to e.g. a sleeve held by a robotic arm by stereotactic imaging, which stereotactic imaging may be a fluoroscopy imaging or an ultrasound imaging. The targeting sleeve or targeting device itself or a rigidly connected structure may serve as reference between multiple C-arm views. With the assumption that the position of the bony structure is relatively stable, movements of the targeting device can then be displayed live relative to the bony structures by simply feeding in the relative position information from a robotic arm, without the need for further imaging. That means, that a targeting task for example for a pin or screw placement can be accurately performed in simple steps of for example acquiring bi- or multi-planar images of the relevant bony structure with a reference body, which may be attached to the robotic arm, positioning by a surgeon a targeting sleeve or targeting device connected to the robot to be aligned with the desired pin or screw trajectory while the relative position is displayed on a screen by the arm movement information coming from position sensors in the robot and the assumption that the bony fragment stays stationary during the process. Then, a verification can be performed that the relative position of targeting device and anatomical structure is now correct through additional imaging.

Robotic arm in this context means a mechanical arm with at least 5 degrees of freedom DoF for the end effector, which may be the targeting device, where relative movements of the tip can be tracked through position sensors in all relevant moving parts and hinges. The hinges or joints of the robotic arm could also be equipped with actuators and/or clutches in order to constrain movements in certain direction and/or block the device position during control imaging.

Stereotactic imaging is calculating the position and orientation of a three dimensional representation of an anatomic structure by correlating for example two fluoroscopic images with a relative angle between them. Since the relative position and orientations by standard C-arms is typically not tracked or known, the invention may utilize a stationary reference body with for example radio-dense markers that are visible in both images. In the targeting application for the femoral head, the femoral head is assumed as spherical. So in this particular case, no underlying CT scan is needed to display the position of a screw relative to the head in the three dimensional image. For application in more general structures, a three dimensional shape estimation may be provided with assumptions about the bone shape by comparing the same with a bone data base. For a more accurate registration of three dimensional structures, a pre- or intra-operative CT scan may be fed in as underlying information, and by identifying the outer contour of structures or fragments and comparing them to the object shape known from the three dimensional scan, the position and orientation may be identified. This may be facilitated by replacing a reference body in the field of view for images. In the approach, this reference body may be connected to the robotic arm or be integrated in the arm.

Thus, the invention provides a highly accurate reproducible process and a device, which may provide a highly accurate and reproducible process to target pins or screws. Especially, if there is an underlying CT, the position of any screw or pin or implant can be accurately planned and executed by the surgeon in the surgery while significantly reducing the need for radiation and potential increase speed of surgery.

For this purpose, the following exemplary embodiments are described along the figures to illustrate the operation of the device for computer assisted surgery and the method for operating the same.

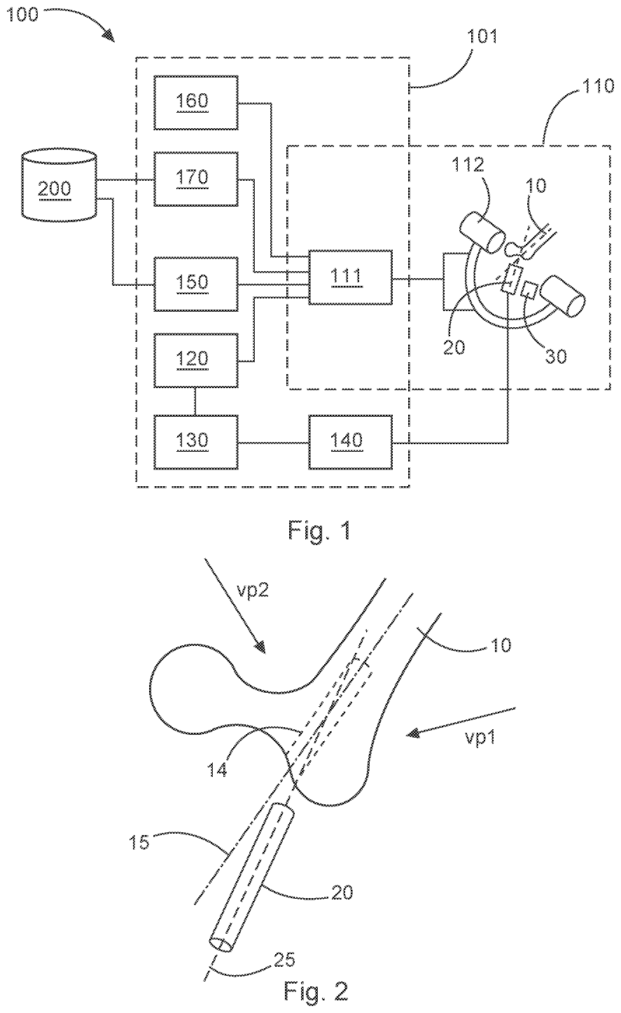

FIG. 1 illustrates a device for computer assisted surgery 100 and the schematic structure thereof. The device for computer assisted surgery 100 may have a processing unit 101 and a field unit for example in form of an imaging device 112, which may be a C-arm x-ray imaging device. The processing unit may have access to a data base 200, in particular a bone data base 200. The entire operation situation, here illustrated as a bony structure 10, a targeting device for an implant 20 and a targeting device for a sub-implant 30 can be imaged by an imaging device 112. It should be noted that for positioning of a single part implant only the targeting device 20 for a single implant is required. In case not only a single implant, but a multiple part implant is to be applied, e.g. a femoral nail and its locking screw, not only a single implant targeting device is required, but an implant targeting device 20 for a main-implant, e.g. the femoral nail, and an implant targeting device 30 for a sub-implant, e.g. its locking screw, is required. It should also be noted, that of course an implant 28 (illustrated in FIG. 8) may be connected to the implant targeting device 20. If required also a here not illustrated sub-implant may be connected to the sub-implant targeting device 30.

An image of the implanting situation, which is taken by the imaging device 112, may be transferred to the processing unit 101. In case that the imaging device 112 acquires more than one image, in particular more than one image from different points of view, the different images may be provided to the processing unit 101. In case a plurality of images are acquired, in particular from different points of view, a correlation unit 111 may correlate the different images, in order to for example combine two or more bi-planar images to a three dimensional illustration of the implanting situation. This image information may then be provided to different units of the processing unit 101. The processing unit 101 may determine based on the acquired imaging information acquired by the image acquiring unit 110 a deviation of an intended implantation axis of the bony structure and an implant trajectory of an implant to be implanted. This deviation may be determined and acquired by the deviation acquiring unit 120. The processing unit 101 may use a determined position, which may be determined by the position determining unit 160 based on the acquired images. The processing unit may also receive supporting information, for example received from an implanting area determining unit 150 and an identification unit for identifying a type of an implant 170. The implanting area determining unit 150 and the identification unit fora type of an implant 170 may receive information for determining the implanting area and the identification of a type of an implant from an external data base 200. The external data base 200 may have included information regarding anatomical geometries. This information regarding anatomical geometries may have included a statistical bone data base where empirical information of different bone geometries are stored, but may also include individual patient related bone data, acquired before.

The deviation acquiring unit 120 may acquire a deviation from the implanting trajectory of the implant targeting device and/or implant and the implanting axis of the implanting area of the patient's bone. Based thereon the motion determining unit 130 may determine the required motion. The motion determining unit 130 provides this information to a motion controlling unit 140, which in turn may control the motion of for example a robot arm (which is not illustrated here) to bring the implantation trajectory 25 related to the implant targeting device and/or implant and the implanting axis 15 of the implanting area 14 of the bony structure 10 into an alignment, as illustrated in FIG. 2.

FIG. 2 illustrates in more detail the situation at the bony structure 10 which has an implanting axis 15 and an implant targeting device 20 and/or implant 28 (not illustrated in FIG. 2) which has an implanting trajectory 25. The implanting axis 15 corresponds to an implanting area 14 of the bony structure 10. An implant targeting device 20 to which a here not illustrated implant 28 may be connected, has an implanting trajectory 25. As can be seen in FIG. 2, the trajectory 25 of the implant targeting device 20 does not correspond to the implanting axis 15 of the bony structure 10. In conventional situations, in such cases a surgeon will relocate the implant targeting device 20 in order to bring the implanting trajectory 25 of the implant targeting device 20 into an alignment to the implanting axis 15 of the bony structure 10. However, as the bony structure 10 on a regular basis is obscured by tissue of the patient 1, a surgeon does not have a full overview on the implanting situation and according to the surgeon's experience it requires more or less steps to iteratively align the implanting trajectory 25 of the implant targeting device 20 and/or implant 28 to the implanting axis 15 of the bony structure 10. To overcome this problem, the present invention provides the device and the corresponding method for operating the device.

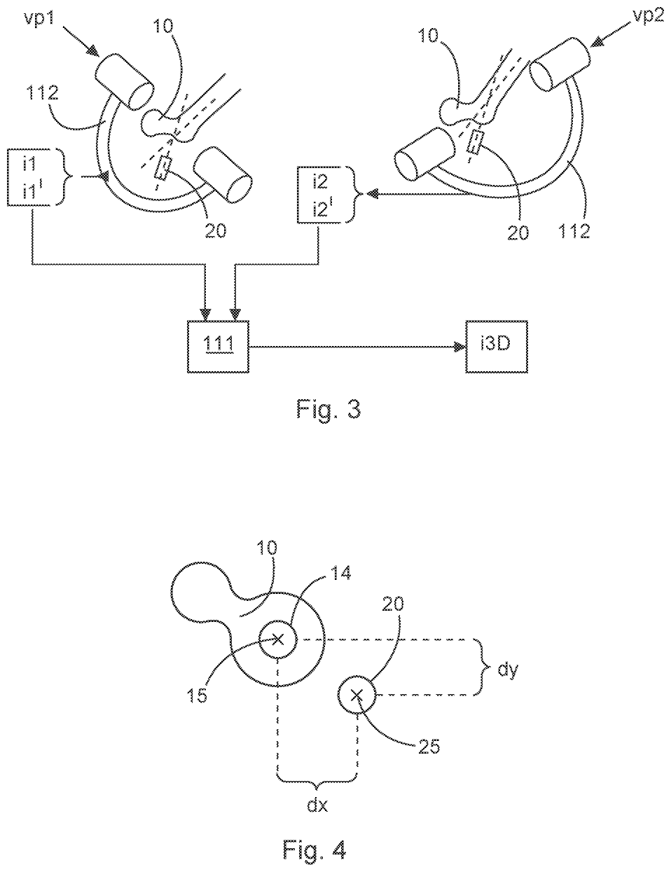

Further, FIG. 2 illustrates two exemplary view points vp1 and vp2 from which for example an x-ray image can be taken. A first bi-planar x-ray image from the first view point vp1 may be correlated to a second bi-planar x-ray image from a second view point vp2 to achieve a three dimensional illustration. This is described in more detail with respect to FIG. 3.

FIG. 3 illustrates a schematic overview of taking a first bi-planar image from a first view point vp1, illustrated on the left-hand side, and a second bi-planar x-ray image from a second view point vp2, illustrated on the right-hand side. As can be seen, the implantation arrangement of the bony structure 10 and the implant targeting device 20 remains unchanged, whereas the C-arm as the imaging device 112 is rotated to allow imaging from different points of view. A first bi-planar image i1 of the bony structure with the implant targeting device and a second bi-planar image i2 of the bony structure with the implant targeting device are fed to the correlation unit 111 which then may generate a three dimensional image i3D. It should be noted, that collection of the images as illustrated in FIG. 3 may take place at the very beginning, which images i1 and i2 may serve as a basis for the method for operating the computer assisted surgery device. Images i1′ and i2′ may also be taken at the end of the procedure in order to monitor the result of the method when operating the device for computer assisted surgery. In some cases, this would permit the procedure to be performed without a collection of additional images besides those collected at the beginning and end. Those images taken at the end i1′ on the left-hand side as well as i2′ on the right-hand side can be also combined at the correlation unit 111 to provide a respective image of a bony structure i3D, in order to monitor, whether the process was conducted sufficiently. This monitoring can be done by the surgeon at the end of the procedure, wherein the surgeon then may decide whether the entire process may be repeated, if needed and desired. Additional images can be acquired and correlated also at any desired time in order to provide a basis for an additional monitoring.

FIG. 4 illustrates a sectional view of the bony structure 10 having illustrated therein the implanting area 14 of the bony structure 10 as well as the implanting axis 15. FIG. 4 also illustrates the implant targeting device 20 and its implanting trajectory 25. The target is that the implanting axis 15 of the bony structure corresponds to the implanting trajectory 25 of the implant targeting device 20 and/or the implant 28. In order to achieve a good implantation, both, implanting axis 15 as well as implanting trajectory 25 should be in alignment, so that the implant 28 which may be connected to the implant targeting device 20 fully aligns with the implanting area 14 of the bony structure 10. In FIG. 4 however, there is a deviation between the both implanting axis 15 and implanting trajectory 25, which deviation is illustrated as a deviation in the x-direction dx and in the y-direction dy. This deviation dx and dy may be acquired by the deviation acquiring unit 120 based on the images acquired by the image acquiring unit 110. Based on this determined deviation, the required motion can be determined by the motion determining unit 130 which in turn serves as a basis for the motion control being conducted by the motion control unit 140. The trajectory 25 of the implant targeting device 20 may be determined based on information which may be acquired from an external data base 200. This external data base 200 may include geometrical information on typical bony structures, so that this information may serve for the implanting area determining unit 150 to determine an optimal implanting area 14 and correspondingly an implanting axis 15. In the same manner, the external data base 200 may provide geometrical information of an implant, which may be provided to the identification unit 170 for identifying a type of an implant. Both units 150 and 170 may provide a geometrical information on the implant axis 15 and a required/desired implanting trajectory 25 to the processing unit 101 in order to determine the deviation from the actual implanting trajectory 25 and the required motion to bring the both aspects, the implanting axis 15 and the implanting trajectory 25 of the implant targeting device 20, into alignment.

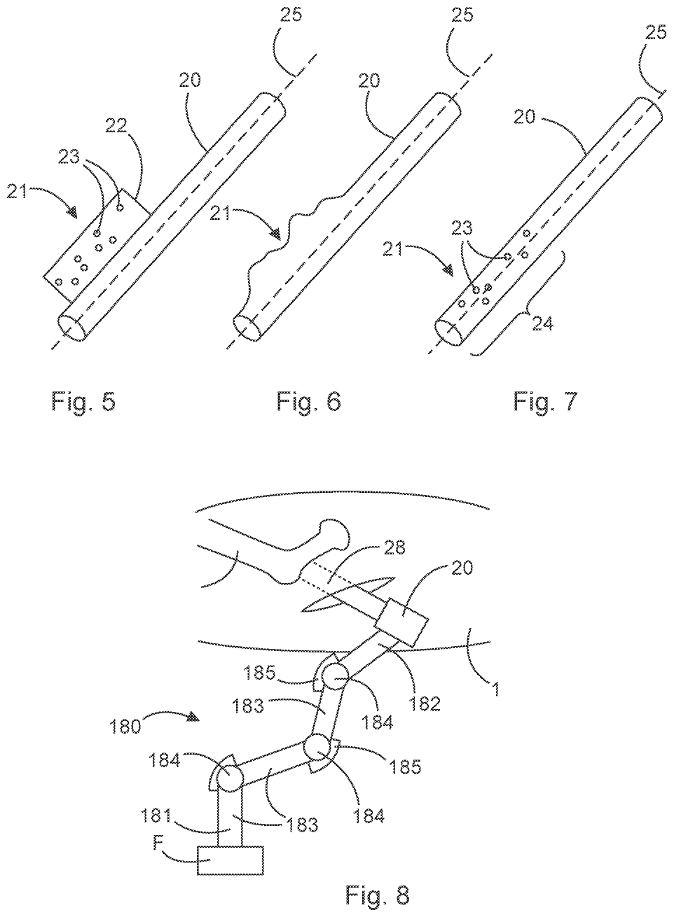

FIG. 5 illustrates an implant targeting device 20 with a reference geometry 21 according to an embodiment of the invention. In order to simplify the determination of the geometrical position and orientation of an implant targeting device 20 and its trajectory 25, a reference body 22 may be provided, which may be attached to the implant targeting device 20. The reference body 22 may also be provided with fiducial markers 23. It should be noted, that instead of the implant targeting device 20, the reference body 22 may also be attached to the implant 28 (not illustrated in FIG. 5), which is to be connected to the implant targeting device 20. However, it should be noted, that the position of an implant 28 which is to be connected to the implant targeting device 20 is fixed and defined with respect to position and orientation, so that attaching the reference body 22 to the implant targeting device 20 may also allow the determination of the position and orientation of the implant 28 which is to be connected to the implant targeting device 20, without having the reference body 22 too close to the bony structure 10.

FIG. 6 illustrates an implant targeting device 20 with a reference geometry 21 according to another embodiment of the invention. As an alternative or in addition to the embodiment illustrated with respect to FIG. 5, the implant targeting device 20 may also have as a reference geometry a unique shape, which shape allows an identification of the position and orientation of the implant targeting device 20 in an x-ray image. FIG. 6 illustrates this by a schematic unique shape of the implant targeting device 20. However, this may require a sufficient contrast of the implant targeting device over the environment in an x-ray image.

FIG. 7 illustrates an implant targeting device 20 with a reference geometry 21 according to yet another embodiment of the invention. As a further alternative, fiducial markers 23 may be provided in a unique pattern directly within the implant targeting device 20 in order to achieve the same purpose as illustrated in the embodiment of FIG. 5. It should be noted, that the unique distribution and pattern of fiducial markers 23 may also be provided directly into the implant 28 which is to be connected to the implant targeting device 20, as the internal fiducial markers 23 when being located within the implant shape may avoid any disturbance when implanting the implant 28 into the implanting area 14. Thus, the reference geometry 24 may be provided as an integral portion of the implant targeting device and as an alternative may also be provided as an integral portion of the implant itself. If providing a reference geometry, e.g. in form of fiducial markers to both, the implant targeting device 20 and the implant 28, also the correct positioning of the implant 28 onto the targeting device 20 may be determined and monitored.

FIG. 8 illustrates a device for computer assisted surgery according to an exemplary embodiment of the invention. The device provides a fix point F to which fix point F the first segmented arm 180 is connected. The first segmented arm 180 is connected to the fix point with a first end 181 of the first segmented arm, wherein a second end 182 is connected to the implant targeting device 20. The segmented arm 180 comprises a plurality of segments 183, which segments 183 are connected at and with joints 184 between the segments. These joints 184 may be brought from a fixed state, where the adjacent segments cannot be moved with respect to each other, into a released state, where one or more of the segments 183 may be moved with respect to each other in at least 1 degree of freedom. Each joint 184 may be provided with sensors in order to detect the position and orientation of the adjacent segments 183 with respect to each other. This position and orientation information may be used to determine the relative position of the implant targeting device 20 with respect to the fixed point F. The joints 184 may also be provided with actuators 185 in order to actuate a movement of adjacent segments 183 along the joints 184. This may allow a surgeon to bring the implant targeting device 20 into a respective position with respect to a bony structure 10 of a patient 1, in order to position the implant 28 correctly into the implanting area 14 of the bony structure 10.

The device as described above with respect to any of the figures, in particular FIG. 1 and FIG. 8 may provide the surgeon with information how to move the segmented arm when the joints are released. For this purpose the device may provide the surgeon with various coordinates and instructions on how far to move the targeting device and/or the implant with respect to the bony structure to archive alignment of the trajectories. The device may monitor the situation continuously, periodically or by incident, i.e. when detecting passing a particular position. The monitoring of the situation may be updated continuously, periodically or by incident on a monitor to show the surgeon the real time position. It is also possible to show the actual position and the calculated position as well as the measure of a deviation. The device may provide the surgeon with a feedback how exact the surgeon meets the calculated position, e.g. by a stepped warning, e.g. by a green light for optimum position or movement, orange light for an acceptable deviation which however should be corrected, and red light for a non-acceptable deviation. Instead of or in addition to an optical information also an acoustic information in form of different tones can be given. The device may also give to the surgeon a haptic feedback, e.g. by generating a recognizable resistance when leaving the calculated position to a certain extend. Then, the device may control the segmented robot arm to bring the joints from the released state into the fixed state in which the deviation of the implanting trajectory of the implant targeting device and the implant is below a threshold and which corresponds to a sufficient implanting situation. The device may determine whether the deviation is below a threshold. The device may compare the calculated coordinates with the actual coordinates continuously, periodically or by incident. The device may use a pre-determined threshold, which may come from a database or may be input manually. The threshold may be based on an implant database or bone database, and may be pre-set to a certain percentage of e.g. bone geometries in the bone database, so that the threshold covers e.g. 95% of all relevant bone geometries. The percentage may vary according to the anatomical position. In case the deviation is above the threshold, the device may re-iterate the process.

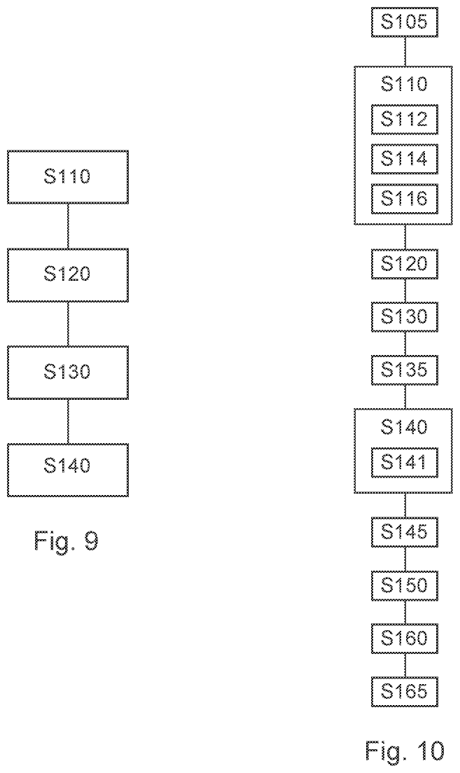

FIG. 9 illustrates the method steps of the method for operating the computer assisted surgery system. The method generally includes acquiring an image, e.g. a three dimensional image S110 and based thereon acquiring a deviation of an implanting trajectory 25 of an implant targeting device 20 or implant 28 from an implanting axis 15 of an implanting area 14 of a bony structure 10, S120. After having acquired a deviation of the implanting trajectory 25 from an implanting axis 15, the method proceeds with determining a measure of a required motion S130 and then with controlling a motion of an implant targeting device S140. Controlling the motion may be achieved by either driving actuators of the device for computer assisted surgery, or by providing instructions to the surgeon how to move the device for computer assisted surgery.

FIG. 10 illustrates the method for operating a computer assisted surgery device in more detail. With this respect, some of the steps illustrated in FIG. 9 may include further sub-steps. With this respect, for example the step of acquiring a three dimensional image S110 may further include acquiring a first and a second bi-planar image S112. Further, step 110 may include a correlation of the first and second bi-planar image S114 and the step of generating a three dimensional image S116. These sub-steps are required, if for example an imaging unit only provides bi-planar images. However, if an image acquiring unit or an imaging device is capable of directly providing a three dimensional image, the step S110 does not mandatorily require the sub-steps S112, S114 and S116. The entire method as described along the flow-chart of FIG. 10 starts with bringing the joints of the segmented arms into a fixed state S105 and then acquiring a three dimensional image S110 as illustrated above. Afterwards, based on the generated three dimensional image, the method proceeds with acquiring a deviation of the implanting trajectory S120 and with determining a measure of a required motion S130. Then, at least one of the joints is brought into a released state S135. It should be noted, that bringing the joint into a released state S135 may also be conducted before step S130 and also before step S120, as the acquisition of the deviation and the determination of the required motion does not depend on the state of the joints. The method then proceeds with controlling a motion of the implant targeting device S140 which may include controlling of the motion of the single segments S141. Afterwards, the joints are brought into a fixed state again in S145. In S150 and S160, a verification can be carried out based on the determined implanting area and the predetermined implanting axis and by again acquiring an image, e.g. a first and second bi-planar image for verification. It should be noted, that S160 may include the same sub-steps as S110 when acquiring a three dimensional image. Afterwards, the deviation of the predetermined implanting trajectory is determined in S165. It should be noted, that the method further proceeds with determination of the required motion and the controlling of the motion of the robot arms as described above, although it is not illustrated in FIG. 10.

### REFERENCE LIST

1 patient

10 bony structure

14 implanting area of bony structure

15 implanting axis

20 (main-)implant targeting device

21 reference geometry of (main-)implant targeting device

22 reference body of (main-)implant targeting device

23 fiducial markers of (main-)implant targeting device

24 reference geometry as integral portion

25 implanting trajectory of (main-)implant targeting device and/or implant

28 (main-)implant

30 (sub-)implant targeting device

100 device for computer assisted surgery

101 processing unit

110 image acquiring unit

111 correlation unit

112 imaging device

120 deviation acquiring unit

130 motion determining unit

140 motion controlling unit

150 implanting area determining unit

160 position determining unit

170 identification unit for identifying a type of an implant

180 (first) segmented arm

181 first end of (first) segmented arm

182 second end of (first) segmented arm

183 segments of (first) segmented arm

184 joints between segments of (first) segmented arm

185 actuators of (first) segmented arm

200 bone data base

dx, dy deviation of the predetermined implanting trajectory

F fixed/reference point, mounting point

i1, i2 first/second (bi-)planar images of bony structure with implant targeting device

i1′, i2′ first/second monitoring/verification images

i3D three dimensional bony structure

vp1, vp2 first/second view point

S105 bringing joints of segmented arm into fixed/locked state

S110 acquiring a three dimensional image

S112 acquiring a first/second (bi-)planar image

S114 correlating first and second (bi-)planar image

S116 generating three dimensional image

S120 acquiring a deviation of implanting trajectory

S130 determining a measure of a required motion

S135 bringing joint into a released state

S140 controlling a motion of implant targeting device

S141 controlling a motion of segments

S145 bringing joint into a fixed state

S150 determining implanting area and predetermined implanting trajectory

S160 acquiring first and second (bi-)planar image for verification

S165 determining deviation of predetermined implanting trajectory

1. A method for operating a computer assisted surgery device, the method comprising:
acquiring an x-ray image of a bony structure together with at least one (1)of an implant targeting device having an implanting trajectory and a reference geometry, and (2) an implant having an implanting trajectory and a reference geometry, the bony structure having an implanting area with a predetermined implanting axis;
acquiring a deviation of the implanting trajectory of at least one of (1) the implant targeting device and (2) the implant from the predetermined implanting axis of the implanting area based on the acquired x-ray image;
determining a measure of a required motion of the at least one of (1) the implant targeting device and (2) the implant to be executed, based on the deviation, for bringing the implanting trajectory of the at least one of (1) the implant targeting device and (2) the implant from a deviated state of the implanting trajectory of the at least one of (1) the implant targeting device and (2) the implant into alignment with the predetermined implanting axis of the implanting area; and controlling a motion of at least one of (1) the implant targeting device and (2) the implant based on the determined measure of required motion.

2. The method according to claim 1, wherein the computer assisted surgery device has a segmented robot arm with a plurality of segments and a plurality of joints, wherein two adjacent segments of the plurality of segments are coupled with a joint of the plurality of joints, the segmented robot arm capable of being transitioned from a fixed state to a released state and vice versa, wherein a first end of the segmented robot arm is connected to a fix point and wherein a second end of the segmented robot arm is connected to the implant targeting device, wherein the method further comprises:
before acquiring the x-ray image, transitioning the plurality of joints of the segmented robot arm into the fixed state, after determining a measure of required motion, transitioning at least one of the plurality of joints to the released state,
controlling a motion of at least one of the segments of the plurality of segments adjacent to the released joints in order to move the at least one of the implant targeting device and the implant according to the determined measure of required motion, and
transitioning the released joints from the released state into the fixed state.

3. The method according to claim 2, wherein the segmented robot arm ) has a plurality of actuators each adapted to controllably actuate a motion of two adjacent segments of the plurality of segments with respect to each other along their connecting joint, wherein the method further comprises:
before acquiring the x-ray image, controlling the plurality of actuators to transition
the plurality of joints of the segmented arm into the fixed state,
after determining a measure of required motion, controlling the plurality of actuators to transition the plurality of joints to the released state and move at least a part of the segments of the plurality of segments to move the at least one of the implant targeting device and the implant according to the determined measure of required
motion, and
controlling the plurality of actuators to transition the plurality of joints of the segmented arm into the fixed state.

4. The method according to claim 1, wherein acquiring
the x-ray image of the bony structure together with at least one of the implant targeting device and the implant comprises:
acquiring a first at least bi-planar image from a first view point onto the bony structure together with the at least one of the implant targeting device and the implant, and a second at least bi-planar image from a second view point onto the bony structure together with the at least one of the implant targeting device and the implant, correlating the first at least bi-planar image and the second at least hi-planar image; and
generating a three dimensional image of the bony structure based on the first at least bi-planar image, the second at least bi-planar image, and a correlation of the first at least bi-planar image and the second at least bi-planar image.

5. The method according to claim 1, further comprising determining the implanting area and the predetermined implanting axis based on the acquired x-ray image of the bony structure and a bone data base having stored therein a plurality of data of bony structures, optimized implanting areas thereon and/or therein, and predetermined implanting axes, as well as correlations thereof.

6. The method according to claim 1, wherein the implanting area of the bony structure represents an area which is defined by a geometry of an implant to be implanted on and/or in the bony structure.

7. The method according to claim 1, wherein the method comprises
after controlling a motion of the at least one of the implant targeting device and the implant, acquiring a further at least bi-planar image from a first view point onto the bony structure together with the at least one of the implant targeting device and the implant, and a further at least bi-planar image from a second view point onto the bony structure together with the at least one of the implant targeting device and the implant, determining a deviation of the implanting trajectory of the at least one of the implant targeting device and the implant from the predetermined implanting axis of the implanting area of the bony structure, and if the deviation is above a predetermined threshold, repeating the step of determining a measure of a required motion of the at least one of the implant targeting device and the implant to be executed for bringing the implanting trajectory of the at least one of the implant targeting device and the implant from an acquired deviation of the implanting trajectory of the at least one of the implant targeting device and the implant into alignment with the implanting axis of the implanting area of the bony structure, and controlling a motion of the at least one of the implant targeting device and the implant.

8. A device for computer assisted surgery, the device comprising:
an image acquiring unit adapted for acquiring an x-ray image of a bony structure together with at least one of an implant targeting device and an implant having an implanting trajectory and a reference geometry;
a deviation acquiring unit adapted for acquiring a deviation of the implanting trajectory of the at least one of the implant targeting device and the implant from a predetermined implanting axis of an implanting area of the bony structure based on the acquired x-ray image;
a motion determining unit adapted for determining a measure of a required motion of the at least one of the implant targeting device and the implant to be executed, based on the deviation, for bringing the implanting trajectory of the at least one of the implant targeting device and the implant from the acquired deviation of the implanting trajectory of the at least one of the implant targeting device and the implant into alignment with the predetermined implanting axis of the implanting area; and a motion controlling unit adapted for controlling a motion of the at least one of the implant targeting device and the implant based on the measure of required motion determined by the motion determining unit.

9. (canceled)

10. The device according to claim 8, further comprising:
a segmented arm with a plurality of segments, wherein at least two adjacent segments of the plurality of segments are coupled with a joint being capable of being transitioned from a fixed state to a released state and vice versa, wherein a first end of the segmented arm is connected to a fix point and a second end of the segmented arm is connectable to at least one of (1) the implant targeting device and (2) the implant,
wherein the image acquiring unit is adapted to acquire the x-ray image in the fixed state of the segmented arm; and
wherein the motion controlling unit is adapted for transitioning the segmented arm from the fixed state to the released state for controlling the motion of the segmented arm based on the measure of the required motion determined by the motion determining unit, and for transitioning the segmented arm from the released state to the fixed state.

11. A device for computer assisted surgery, the device comprising:
a first segmented robot arm having a plurality of segments and a plurality of actuators each being adapted to controllably actuate a motion of two adjacent segments with respect to each other about a connecting joint of the two adjacent segments, wherein the connecting joint is capable of being transitioned from a fixed state to a released state and vice versa, wherein a first end of the the first segmented robot arm is connected to a fix point and a second end of the first segmented robot arm is connectable to at least one of (1) an implant targeting device having an implanting trajectory and a reference geometry, and (2) an implant having an implanting trajectory and a reference geometry,
an image acquiring unit adapted for acquiring an x-ray image of a bony structure together with the at least one of the implant targeting device and the implant at a fixed state of the first segmented robot arm;
a deviation acquiring unit adapted for acquiring a deviation of the implanting trajectory of the at least one of the implant targeting device and the implant from a predetermined implanting axis of an implanting area of the bony structure based on the acquired x-ray image;
a motion determining unit adapted for determining a measure of a
required motion of the at least one of the implant targeting device and the implant to be executed, based on the deviation, for bringing the implanting trajectory of the at least one of the implant targeting device and the implant from the acquired deviation of the implanting trajectory of the at least one of the implant targeting device and the implant into alignment with the predetermined implanting axis of the implanting area; and
a motion controlling unit adapted for transitioning connecting joints of the first segmented robot arm from the fixed state to the released state, for controlling the motion of the plurality of actuators of the first segmented robot arm based on the measure of required motion determined by the motion determining unit, and for transitioning the connecting joints of the first segmented robot arm from the released state to the fixed state.

12. The device according to claim 8, wherein the image acquiring unit is adapted for acquiring a first at least bi-planar image from a first view point onto the bony structure together with at least one of an implant targeting device and an implant, and a second at least bi-planar image from a second view point onto the bony structure together with the at least one of the implant targeting device and the implant, and for composing an x-ray image out of the first at least bi-planar image and the second at least bi-planar image.

13. The device according to claim 11, wherein the image acquiring unit comprises a correlation unit adapted for correlating the first at least bi-planar image and the second at least bi-planar image and generating a three dimensional image of the bony structure based on the first at least bi-planar image, the second at least bi-planar image, and the correlation of the first at least bi-planar image and the second at least bi-planar image.

14. The device according to claim 8, further comprising an implanting area determining unit being adapted for determining the implanting area and the predetermined implanting axis based on the acquired bony structure and a bone data base having stored therein a plurality of data of bony structures, optimized implanting areas thereon and/or therein, and predetermined implanting axes, as well as a correlation thereof.

15. The device according to claim 8, further comprising as part of a system a reference geometry, wherein the reference geometry is a reference body which is attachable to at least one of the implant targeting device and the implant, representing a unique position and orientation of the at least one of the implant targeting device and the implant.

16. The device according to claim 8, further comprising as part of a system an implant targeting device, wherein the reference geometry is an integral portion of the implant targeting device, wherein the integral portion has a geometry having a unique projection pattern for each projection direction.

17. The device according to claim 8, further comprising as part of a system an implant, wherein the reference geometry is an integral portion of the implant, wherein the integral portion has a geometry having a unique projection pattern for each projection direction.

18. The device according to claim 8, wherein the reference geometry has a plurality of fiducial markers, wherein the fiducial markers have a spatial arrangement having a unique projection pattern for each projection direction.
